(19) United States
(12) Patent Application Publication
(10) Pub. No.: US 2018/0185440 A1
(43) Pub. Date: Jul. 5, 2018

(54) PHARMACEUTICAL COMPOSITIONS AND METHODS FOR DELIVERY OF BREMELANOTIDE

(71) Applicant: NOVEN PHARMACEUTICALS, INC., Miami, FL (US)

(72) Inventors: Masayuki Suzuki, Miami, FL (US); Joshua A. Eldridge, Miami, FL (US)

(73) Assignee: NOVEN PHARMACEUTICALS, INC., Miami, FL (US)

(21) Appl. No.: 15/857,213

(22) Filed: Dec. 28, 2017

Related U.S. Application Data
(60) Provisional application No. 62/440,084, filed on Dec. 29, 2016.

(51) Int. Cl. A61K 38/12; A61K 47/18; A61K 47/14; A61K 9/70; A61K 9/48; A61P 15/10

(52) U.S. Cl. CPC A61K 38/12; A61K 47/18; A61K 47/14; A61P 15/10; A61K 9/4866; A61K 9/4858; A61K 9/7084

(57) ABSTRACT

Described are topical, transdermal and transmucosal pharmaceutical compositions comprising bremelanotide that include bremelanotide or a pharmaceutically acceptable salt thereof and a permeation enhancer, in any form suitable for topical, transdermal or transmucosal delivery, including transmucosal delivery through the gastrointestinal tract after oral administration. Also provided are methods of making and using the pharmaceutical compositions.

### CROSS-REFERENCE TO RELATED APPLICATIONS

This application claims the benefits under 35 U.S.C. § 119(e) to U.S. provisional application 62/440,084, filed Dec. 29, 2016, the contents of which are incorporated herein by reference in their entirety.

### FIELD

Described herein are pharmaceutical compositions comprising bremelanotide or a therapeutically acceptable salt thereof for topical, transdermal, or transmucosal delivery, including transmucosal and transdermal patches and pharmaceutical capsules containing them. Methods of making and using the compositions also are described.

### BACKGROUND

Medical use of protein drugs is constrained by major drawbacks. Oftentimes, the short biological half-life of protein drugs requires frequent administration. Additionally, protein drugs may undergo rapid degradation in mucosal tissues and/or in the gastrointestinal tract. Such degradation limits the bioavailability and efficacy of orally administered protein drugs. Therefore, the most common mode of protein drug administration is by injection.

However, administration by injection suffers from its own drawbacks, such as significant inconvenience to patients, particularly for “on-demand/as-needed” drugs and drugs that need to be administered daily. Moreover, administration by injection requires delivery systems (e.g., syringes), adding to the costs of manufacturing, dispensing and administering the drugs.

Bremelanotide is a cyclic peptide with a molecular weight of 1,025 g/mol. A formulation of bremelanotide for subcutaneous injection is being developed for the treatment of Hypoactive Sexual Desire Disorder (HSDD) in premenopausal females. For this therapeutic indication, a dose of 1.75 mg is to be taken “on-demand/as-needed” and has a 30 to 60 minute onset period. Other uses of bremelanotide include the treatment of erectile dysfunction, inflammatory diseases, hemorrhagic shock, and reperfusion injury (ischemia). Yet further uses include the treatment of aphthous stomatitis and arthritis, and the prevention of acute kidney failure as may occur following surgery.

There is a need, therefore, for compositions and methods for administering bremelanotide by alternative routes of administration.

### SUMMARY

In accordance with some embodiments there are provided pharmaceutical compositions for topical, transdermal or transmucosal delivery of bremelanotide, comprising bremelanotide or a pharmaceutically acceptable salt thereof and a penetration enhancer, wherein the penetration enhancer comprises one or more selected from:

(a) a compound of Formula I

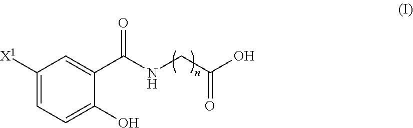

wherein X¹ is H or Cl and n is an integer from 6 to 21, or a pharmaceutically acceptable salt thereof;
(b) an N-substituted bis-3,6-[4-aminobutyl]-2,5-diketopiperazine or a pharmaceutically acceptable salt thereof;
(c) an alkyl glucoside or a pharmaceutically acceptable salt thereof;
(d) lauroyl-L-carnitine; and
(e) dimethyl palmitoyl ammonio propanesulfonate or a pharmaceutically acceptable salt thereof.

In some embodiments, the penetration enhancer comprises one or more selected from N-(8-[2-hydroxybenzoyl]amino)caprylic acid (SNAC), N-(5-chlorosalicyloyl)-8-aminocprylic acid (5-CNAC), N-(10[2-hydroxybenzoyl]amino)decanoic acid (SNAD), bis-3,6(4-fumarylaminobutyl)-2,5-diketopiperazine, n-dodecyl-β-D-maltoside, lauroyl-L-carnitine, dimethyl palmitoyl ammonio propanesulfonate, and pharmaceutically acceptable salts of any one or more thereof. In some embodiments, the penetration enhancer comprises N-(8-[2-hydroxybenzoyl]amino)caprylic acid (SNAC). In some embodiments, the penetration enhancer comprises n-dodecyl-β-D-maltoside.

In some embodiments, the penetration enhancer constitutes from about 0.1% to about 99.9% by weight of the composition. In some embodiments, the weight ratio of penetration enhancer to bremelanotide or pharmaceutically acceptable salt thereof is from about 999:1 to about 1:999.

In some embodiments, the composition further comprises a pharmaceutically acceptable carrier for topical, transdermal or transmucosal administration comprising one or more selected from a mucoadhesive polymer, a pressure-sensitive adhesive polymer, and water. In some embodiments, the pharmaceutically acceptable carrier comprises a mucoadhesive polymer comprising one or more selected from hydroxypropylmethylcellulose (HPMC), polyvinylpyrrolidone, poly(2-hydroxyethyl methacrylate) (PHEMA), carbomers, carboxyvinyl polymers, poly(methacrylic acid), polyacrylic acid, thiolated poly(acrylic acid), ethylhydroxyethylcellulose (EHEC), carboxymethylcellulose (CMC), a salt of carboxymethylcellulose, hydroxypropylcellulose (HPC), hydroxyethylcellulose (HEC), hydroxyethylmethylcellulose, polyvinyl alcohol, karaya gum, arabic gum, shiraz gum, xanthan gum, guar gum, gellan gum, alginate, sodium alginate, chitosan, thiolated chitosan, hyaluronate, sodium hyaluronate, pectin, gelatin, and veegum. In some embodiments, the pharmaceutically acceptable carrier comprises one or more selected from HPMC, HEC, HPC, PHEMA, PVP, PVA and karaya gum.

In some embodiments, there are provided transdermal or transmucosal patches comprising a composition as described herein in a flexible, finite form wherein the pharmaceutically acceptable carrier comprises a mucoadhesive polymer or a pressure-sensitive adhesive polymer. In some embodiments, the composition is provided as a drug-containing layer and the patch further comprises a backing layer or coating. In some embodiments, the backing comprises one or more selected from ethylcellulose, polyester, polyethylene, vinyl acetate resins, ethylene/vinyl acetate copolymers, polymethacrylates, methacrylic acid-methyl methacrylate copolymers, polyvinyl chloride, polyurethane, foil, non-woven fabric, and cloth. In some embodiments, the backing is a backing layer that is coterminous with the perimeter of the drug-containing layer. In some embodiments, the backing is a backing layer that is a backing layer that extends beyond the outer perimeter of the drug-containing layer in one or more or all directions. In some embodiments, the backing is a dissolvable backing that dissolves in the oral or gastrointestinal environment.

In some patch embodiments, the composition is substantially free of plasticizers other than enhancers (a) through (e).

In some patch embodiments, the patch is a transmucosal patch, and the pharmaceutically acceptable carrier comprises a mucoadhesive polymer. In some patch embodiments, the patch is a transdermal patch, and the pharmaceutically acceptable carrier comprises a pressure-sensitive adhesive polymer. In some patch embodiments, the patch comprises an amount of bremelanotide or pharmaceutically acceptable salt thereof effective to deliver a therapeutically effective amount thereof.

In some embodiments, there are provided pharmaceutical capsules containing a composition as described herein or a patch as described herein. In some embodiments, a capsule may contain a plurality of patches as described herein. In some embodiments, a capsule may release the bremelanotide or pharmaceutically acceptable salt thereof in the gastrointestinal tract. In some embodiments, a capsule comprises an amount of bremelanotide or pharmaceutically acceptable salt thereof effective to deliver a therapeutically effective amount thereof.

In some embodiments, there are provided methods for administering bremelanotide or pharmaceutically acceptable salt thereof to a subject in need thereof, comprising orally administering a composition, patch, or capsule as described herein to a subject in need thereof. In some embodiments, the administration comprises administration to one or more of the oral, ocular, nasal, buccal, vaginal, rectal, or gastrointestinal mucosa of the subject.

In some embodiments, there are provided compositions, patches, or capsules as described herein for administering bremelanotide or pharmaceutically acceptable salt thereof to a subject in need thereof.

In some embodiments, there are provided uses of bremelanotide or pharmaceutically acceptable salt thereof, in the preparation of a composition, patch, or capsule as described herein, for administering bremelanotide or pharmaceutically acceptable salt thereof to a subject in need thereof.

In some embodiments, the subject is suffering from or at risk of developing Hypoactive Sexual Desire Disorder (HSDD), erectile dysfunction, inflammatory disease, hemorrhagic shock, reperfusion injury (ischemia), aphthous stomatitis, arthritis, and/or acute kidney failure.

### BRIEF DESCRIPTION OF THE DRAWINGS

FIGS. 1A-1C illustrate drug delivery patches and a capsule as described herein. In the embodiment shown in FIG. 1A, the patch comprises a drug-containing adhesive layer and a drug-impermeable backing layer that is coterminous with the active surface area of the adhesive layer. In the embodiment shown in FIG. 1B, the patch comprises a drug-containing adhesive layer and a drug-impermeable backing layer that is coterminous with the active surface area of the a drug-containing adhesive layer, and is provided with a release liner. In the embodiment shown in FIG. 1C, a pharmaceutical capsule contains a plurality of patches.

FIGS. 2A-B illustrate the delivery of bremelanotide (“BMT”; μg/cm²) from an aqueous bremelanotide composition as described herein as compared to a control composition in an in vitro transdermal model using human skin samples. FIG. 2A provides the results from one tissue donor (“Donor 1”) while FIG. 2B provides results from a different tissue donor (“Donor 2”).

FIGS. 3A-B illustrate the delivery of bremelanotide from an aqueous bremelanotide composition as described herein as compared to a control composition in an in vitro buccal tissue model, where FIG. 3A reports BMT flux (μg/cm²/hr) and FIG. 3B provides cumulative BMT delivery (μg/cm²).

FIGS. 4A-B illustrate the delivery of bremelanotide from transmucosal drug delivery patches as described herein in an in vitro buccal tissue model, where FIG. 4A reports BMT flux (μg/cm²/hr) and FIG. 4B provides cumulative BMT delivery (μg/cm²).

FIG. 5 illustrates blood levels (pg/ml) of bremelanotide achieved in rats by oral administration of capsules containing either a bremelanotide composition (Example 4) or transmucosal drug delivery patches as described herein (Example 5) as compared to a capsule containing 100% bremelanotide acetate (Control 2).

FIG. 6 illustrates blood levels (pg/ml) of bremelanotide achieved in rats by oral administration of capsules containing transmucosal drug delivery patches as described herein (with different amounts of sodium N-(8-[2-hydroxybenzoyl]amino)caprylate (SNAC) in the mucoadhesive layer), illustrating that the amount of SNAC in the mucoadhesive layer impacts the drug delivery profile.

FIG. 7 illustrates blood levels (pg/ml) of bremelanotide achieved in rats by oral administration of capsules containing transmucosal drug delivery patches as described herein (with different amounts of SNAC in the mucoadhesive layer), further illustrating that the amount of SNAC in the mucoadhesive layer impacts the drug delivery profile.

FIG. 8 illustrates blood levels (pg/ml) of bremelanotide achieved in rats by oral administration of capsules containing transmucosal drug delivery patches as described herein. In FIG. 8, Example 14 includes n-dodecyl-β-D-maltoside (n-DMS) in the mucoadhesive layer and Example 15 includes a mixture of n-DMS and SNAC in the mucoadhesive layer.

FIG. 9 illustrates blood levels (pg/ml) of bremelanotide achieved in dogs by oral administration of capsules containing a bremelanotide composition (Example 16) or transmucosal drug delivery patches as described herein (Examples 17 and 18, where the capsule of Example 17 includes an enteric coating on the capsule).

### DETAILED DESCRIPTION

Described herein are pharmaceutical compositions for the topical, transdermal or transmucosal delivery of bremelanotide, comprising bremelanotide or a pharmaceutically acceptable salt thereof and a penetration enhancer. The composition may be in any form suitable for topical, transdermal or transmucosal delivery, including transmucosal delivery through gastrointestinal mucosa after oral administration. In some embodiments the composition is formulated in a non-finite form for topical administration (such as a solution, gel, emulsion, cream, plaster, etc.). In some embodiments, the composition is formulated in a flexible, finite form (e.g., a patch or film) for application to a skin or mucosal surface. In some embodiments, a plurality of patches are contained in a capsule for transmucosal delivery, such as through gastrointestinal mucosa. In some embodiments the composition is formulated in a non-finite form (e.g., powder or granules, optionally provided in capsules) for transmucosal delivery. In some embodiments, the composition further comprises a pharmaceutically acceptable carrier for topical, transdermal or transmucosal administration, such as a pressure-sensitive adhesive or mucoadhesive polymer, water, or other pharmaceutically acceptable carrier for topical, transdermal or transmucosal administration. Also described are methods of making and using such compositions.

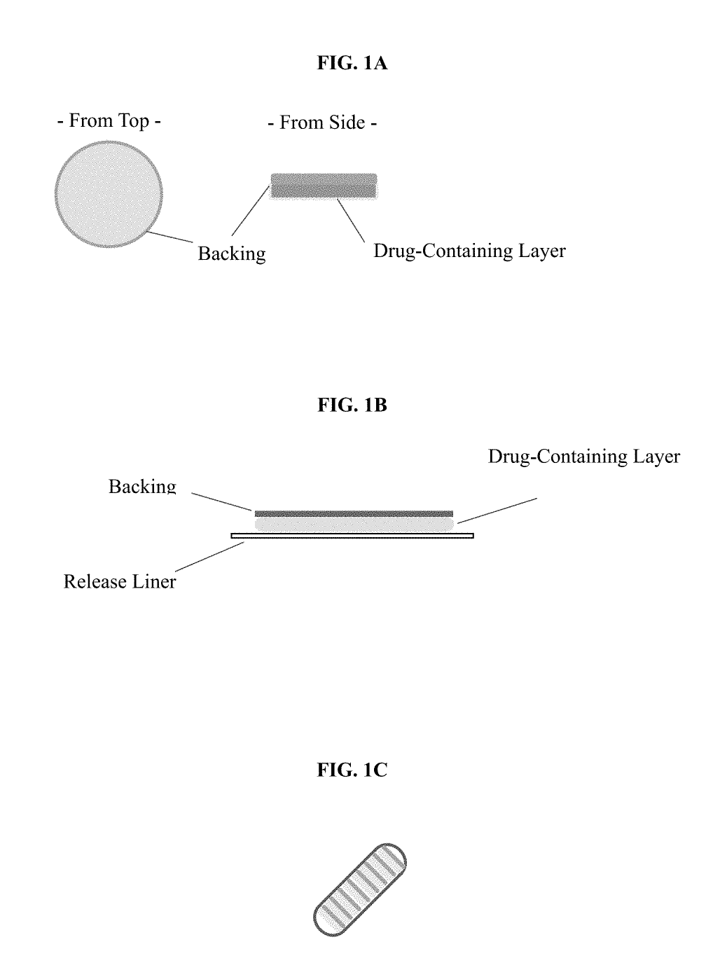

FIGS. 1A-1B illustrate specific embodiments of drug delivery patches as described herein comprising a drug-containing layer and a backing layer. In the embodiment shown in FIG. 1A, the backing layer is coterminous with the active surface area of the drug-containing layer. FIG. 1B shows a similar provided with a release liner. FIG. 1C illustrates an oral pharmaceutical capsule containing a plurality of patches.

### Definitions

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents, and the like to which reference is made in the following description and examples are obtainable from commercial sources, unless otherwise noted.

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein “subject” denotes any animal in need of bremelanotide therapy, including humans. For example, a subject may be suffering from or at risk of developing one or more of hypoactive sexual desire disorder (HSDD), erectile dysfunction, an inflammatory disease, hemorrhagic shock, reperfusion injury (ischemia), aphthous stomatitis, arthritis, acute kidney failure such as may occur following surgery, or may be taking bremelanotide for health maintenance purposes, or for other purposes.

The phrase “substantially free of” as used herein means that the described composition (e.g., mucoadhesive layer) includes less than about 5%, less than about 3%, or less than about 1% by weight, based on the total weight of the composition at issue, of the excluded component(s). The phrase “free of” as used herein means that the described composition (e.g., mucoadhesive layer) is formulated without the excluded component(s), although small amounts may be present as an impurity, contaminant, etc.

As used herein, “active surface area” means the surface area of the drug-containing layer of a transdermal or transmucosal drug delivery patch, and may be independent of the surface area of the backing layer.

As used herein, the terms “topical,” “transdermal” and “transmucosal” refer to delivery, administration or application of a drug by means of direct contact with skin (transdermal) or mucosa (transmucosal) for local or systemic effect. “Topical” application refers to application to a skin or mucosal surface. “Transdermal” delivery includes dermal and percutaneous administration. “Transmucosal” delivery includes passage through any mucosa, including oral, buccal, sublingual, nasal, gastrointestinal, rectal, vaginal and ocular mucosa. In specific embodiments, the compositions described herein may be administered orally for transmucosal delivery through oral, buccal, sublingual, and/or gastrointestinal mucosa.

The term “buccal” is used herein in accordance with its usage in the art of pharmaceutical drug delivery, and includes any surface inside the mouth or upper throat, such as the cheek, inner cheek lining, gums, palate, tongue, tonsils, periodontal tissue, lips, and/or the mucosa of the mouth and pharynx.

As used herein, a transdermal or transmucosal “drug delivery patch” refers to a composition in flexible, finite form that delivers bremelanotide (or a pharmaceutically acceptable salt thereof) through the skin or mucosa upon application to the skin or mucosa, respectively. A transdermal or transmucosal drug delivery patch comprises a drug-containing layer and, optionally a backing. In some embodiments, the drug-containing layer comprises a pressure-sensitive adhesive or mucoadhesive, and is adapted for direct application to a user's (e.g., a subject's) skin or mucosa. In other embodiments, the patch is provided with a separate adhesion means (such as a separate adhesive layer) for application and adherence to the user's skin or mucosa. In some embodiments, the patch conforms to the surface of the skin or mucosa to which it is applied or comes into contact. In some embodiments, the patch maintains contact with the skin or mucosa sufficient to effect drug delivery over the intended delivery period. In some embodiments, the patch remains substantially intact during the intended delivery period. In some embodiments, the patch comprises a release liner that is removed prior to use.

As used herein, something is an “adhesive” if it has the properties of an adhesive per se, or if it functions as an adhesive by the addition of tackifiers, plasticizers, crosslinking agents, or other additives. Thus, in some embodiments, the drug-containing layer comprises a pressure-sensitive adhesive polymer or a mucoadhesive polymer, with bremelanotide or a pharmaceutically acceptable salt thereof dissolved and/or dispersed therein. The drug-containing layer also may comprise tackifiers, plasticizers, crosslinking agents or other additives, such as those described in more detail below.

As used herein, the phrases “therapeutically effective amount” and “therapeutic level” mean that drug dosage or plasma concentration in a subject, respectively, that provides the specific pharmacological response for which the bremelanotide (or pharmaceutically acceptable salt thereof) is administered to a subject in need of such treatment, for whatever reason. It is emphasized that a therapeutically effective amount or therapeutic level of bremelanotide will not always be effective in treating the target disease or condition in a given subject, even though such amount is deemed to be a therapeutically effective amount by those of skill in the art. Those skilled in the art can determine and adjust such amounts in accordance with standard practices as needed to treat a specific subject and/or condition/disease.

In the following description, details and specific embodiments are set forth for purposes of illustration and explanation and not limitation. It will be apparent to those skilled in the art that the invention may be practiced in other embodiments that depart from the details and specific embodiments without departing from the invention.

### Pharmaceutical Bremelanotide Compositions

The pharmaceutical bremelanotide compositions described herein comprise bremelanotide or a pharmaceutically acceptable salt thereof, a penetration enhancer, and, optionally, a pharmaceutically acceptable carrier. For simplicity, in the discussion that follows, the term “bremelanotide” is used to refer to bremelanotide as well as pharmaceutically acceptable salts thereof.

### Bremelanotide

Bremelanotide is a peptide drug having the chemical formula C₅₀H₆₈N₁₄O₁₀, and a net molecular weight of 1025.18 with the chemical name: (3S,6S,9R,12S,15S,23S)-15-[[(2S)-2-acetamidohexanoyl]amino]-9-benzyl-6-[3-(diaminomethylideneamino)propyl]-12-(1H-imidazol-5-ylmethyl)-3-(1H-indol-3-ylmethyl)-2,5,8,11,14,17-hexaoxo-1,4,7,10,13,18-hexazacyclotricosane-23-carboxylic acid and the structure:

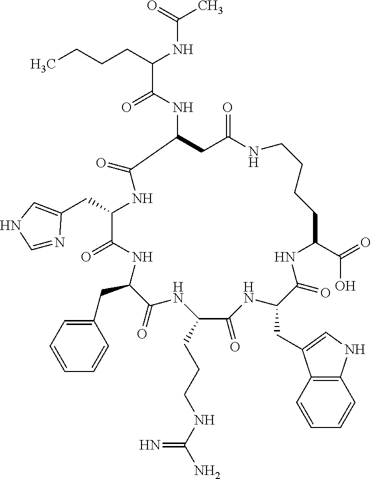

and also is referred to as Ac-Nle-cyclo(-Asp-His-D-Phe-Arg-Trp-Lys)-OH.

As used herein, “pharmaceutically acceptable salt” is used its accordance with its usage in the pharmaceutical arts. Pharmaceutically acceptable salts are well known in the art and described in, for example, S. M. Berge et al., J. Pharmaceutical Sciences, 66:1 (1977). For example, bremelanotide may be prepared and formulated as an acid addition salt, such as a hydrochloride, hydrobromide, sulfide, phosphate, acetate, trifluoroacetate, maleate, citrate, tartrate, oxalate, succinate or mesylate salt. In some embodiments, the compositions comprise bremelanotide acetate.

Bremelanotide (and/or pharmaceutically acceptable salts thereof) and other compounds useful in the compositions described herein may exist as solvates, including hydrates and organic solvates. Hydrates may form during manufacture of a compound or composition, or may form over time due to the hygroscopic nature of the compound. Examples of organic solvates include DMF, ether, and alcohol solvates.

As noted above, for simplicity, in the discussion that follows, the term “bremelanotide” is used to refer to bremelanotide as well as pharmaceutically acceptable salts thereof.

The compositions described herein are formulated with an amount of bremelanotide effective to achieve the target drug delivery, such as an amount effective to achieve the delivery of a therapeutically effective amount of bremelanotide for the intended therapeutic effect. In some embodiments, the composition includes from about 0.1% to about 99.9% by dry weight of bremelanotide, depending on the form in which the composition is formulated (topical, transdermal, transmucosal, finite, non-finite, etc.). Thus, a composition may include an amount of bremelanotide, based on the dry weight of the composition, of about 0.1%, about 0.25%, about 0.5%, about 0.75%, about 1%, about 2%, about 3%, about 4%, about 5%, about 6%, about 8%, about 10%, about 15%, about 20%, about 25%, about 30%, about 35%, about 40%, about 45%, about 50%, about 55%, about 60%, about 65%, about 70%, about 75%, about 80%, about 85%, about 90%, about 93%, about 95%, about 97%, about 99%, or about 99.9%, or any value or range including and in between any of these values.

### Penetration Enhancers

Penetration enhancers that promote the transdermal and mucosal absorption of drugs are known. However, identifying a penetration enhancer effective for a given drug can be an unpredictable undertaking. The inventors surprisingly determined that the follow enhancers are particularly effective for the transdermal or transmucosal delivery of bremelanotide:

(a) a compound of formula I

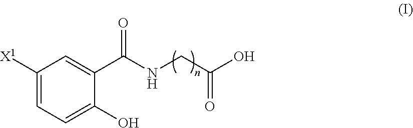

wherein X¹ is H or Cl and n is an integer from 6 to 21, or a pharmaceutically acceptable salt thereof,
(b) a bis-3,6-[4-aminobutyl]-2,5-diketopiperazine where one or both of the 4-aminobutyl groups is substituted at the nitrogen (an “N-substituted bis-3,6-[4-aminobutyl]-2,5-diketopiperazine”) or a pharmaceutically acceptable salt thereof;
(c) an alkyl glucoside or a pharmaceutically acceptable salt thereof;
(d) lauroyl-carnitine (including lauroyl-L-carnitine); and
(e) dimethyl palmitoyl ammonio propanesulfonate.
In some embodiments, the composition includes one or more of enhancers (a)-(e).

Examples of N-substituted bis-3,6-[4-aminobutyl]-2,5-diketopiperazines (b) include those set forth in U.S. Pat. Nos. 5,352,461, 5,503,852, 6,071,497, and 6,331,318, each of which is incorporated by reference herein in their entireties for any and all purposes. Specific examples include:
N-(5-chlorosalicyloyl)-8-aminocaprylic acid,
N-(10-[2-hydroxybenzoyl]amino)decanoic acid,
N-(8-[2-hydroxybenzoyl]amino)caprylic acid,
bis-3,6(4-fumarylaminobutyl)-2,5-diketopiperazine (FDKP),
n-dodecyl-β-D-maltoside,
bis-3,6(4-fumarylaminobutyl)-2,5-diketopiperazine,
dimethyl palmitoyl ammonio propanesulfonate,
   and pharmaceutically acceptable salts of any one or more thereof.

Examples of pharmaceutically acceptable salts of N-(8-[2-hydroxybenzoyl]amino)caprylic acid include:
sodium N-(8-[2-hydroxybenzoyl]amino)caprylate (also known as salcaprozate sodium or SNAC),
8-(N-2-hydroxy-5-chlorobenzoyl)-amino-caprylic acid (5-CNAC) and
N(10-[2-hydroxybenzoyl]amino)decanoic acid (SNAD).

Examples of an alkyl glucoside enhancer (c) include maltosides. Particular examples of maltosides include, but are not limited to:
n-decyl-β-D-maltoside (also known as n-decyl-β-D-maltopyranoside),
n-dodecyl-β-D-maltoside (also known as n-dodecyl-β-D-maltopyranoside or n-DMS), and
6-cyclohexyl-1-hexyl-β-D-maltoside (also known as 6-cyclohexyl-1-hexyl-β-D-maltopyranoside).

The compositions described herein may additionally or alternatively include a penetration enhancer selected from polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin; polar solvents such as dimethyldecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide; salicylic acid; amino acids; benzyl nicotinate; and higher molecular weight aliphatic surfactants such as lauryl sulfate salts; oleic and linoleic acids, ascorbic acid, panthenol, butylated hydroxytoluene, tocopherol, tocopheryl acetate, tocopheryl linoleate, propyl oleate, and isopropyl palmitate. However, in some embodiments, the bremelanotide compositions do not include additional enhancers other than one or more of enhancers (a)-(e) described above.

The penetration enhancer is formulated in a composition as described herein in an amount effective to promote bremelanotide delivery through the skin or mucosa, while maintaining acceptable physical properties of the composition (where applicable). In some embodiments, one or more penetration enhancers selected from (a)-(e) above is included in an amount of from about 0.1% to about 99.9.% by weight of the composition. When more than one permeation enhancer is used, the total amount of penetration enhancers included may be from about 0.1% to about 99.9.% by weight of the composition. Thus, the total amount of penetration enhancer present in the compositions described herein may be about 0.1%, about 0.25%, about 0.5%, about 0.75%, about 1%, about 2%, about 3%, about 4%, about 5%, about 6%, about 8%, about 10%, about 15%, about 20%, about 25%, about 30%, about 35%, about 40%, about 45%, about 50%, about 55%, about 60%, about 65%, about 70%, about 75%, about 80%, about 85%, about 90%, about 93%, about 95%, about 97%, about 99%, or about 99.9%, based on the dry weight of the composition, or any value or range including and in between any of these values. In some embodiments, the weight ratio of penetration enhancer to bremelanotide in the composition may be from about 999:1 to about 1:999, or any value or range including and in between these values.

In some embodiments, the composition is in the form of a transmucosal drug delivery patch (also referred to herein as a “mucoadhesive patch”) comprising a drug-containing mucoadhesive layer comprising bremelanotide and the penetration enhancer, and the penetration enhancer is included in an amount of at least about 5% by dry weight of the mucoadhesive layer, such as from about 5% to about 80% by dry weight of the mucoadhesive layer. Thus, the total amount of enhancer present in the mucoadhesive layer based on the dry weight of the mucoadhesive layer may be about 5%, about 10%, about 15%, about 20%, about 25%, about 30%, about 35%, about 40%, about 45%, about 50%, about 55%, about 60%, about 65%, about 70%, about 75%, about 80%, or any value or range including and in between any of these values.

In some embodiments, composition is in the form of a transdermal drug delivery patch (also referred to herein as a “transdermal patch”) comprising a drug-containing pressure-sensitive adhesive layer comprising bremelanotide and the penetration enhancer, and the penetration enhancer is included in an amount of at least about 5% by dry weight of the pressure-sensitive adhesive layer, such as from about 5% to about 80% by dry weight of the pressure-sensitive adhesive layer. Thus, the total amount of permeation enhancer present in the pressure-sensitive adhesive layer based on the dry weight of the pressure-sensitive adhesive layer, may be about 5%, about 10%, about 15%, about 20%, about 25%, about 30%, about 35%, about 40%, about 45%, about 50%, about 55%, about 60%, about 65%, about 70%, about 75%, about 80%, or any value or range including and in between any of these values.

The inventors surprisingly discovered that permeation enhancers (a)-(e) are particularly useful in a mucoadhesive composition (such as a mucoadhesive patch), because they also can act as a plasticizer. That is, the use of one or more of enhancers (a)-(e) in a mucoadhesive patch may increase the adhesiveness of the patch to mucosa and/or improve the flexibility properties of the patch. Thus, while a mucoadhesive composition or patch may include one or more compounds with plasticizing properties, in some embodiments the mucoadhesive composition or patch is substantially free of or free of other components with plasticizing properties. In specific embodiments, the mucoadhesive composition is in the form of a mucoadhesive layer of a mucoadhesive patch that contains less than about 5%, less than about 1%, less than about 0.1%, less than about 0.05%, less than about 0.01%, or less than about 0.001% by dry weight of a compound with plasticizing properties other than one or more of enhancers (a)-(e).

Because a mucoadhesive composition formulated with one or more of enhancers (a)-(e) does not require another compound with plasticizing properties, the mucoadhesive composition can include more bremelanotide, mucoadhesive polymer, permeation enhancer, and/or other excipients than would be possible if the mucoadhesive composition had to include another compound with plasticizing properties.

### Mucoadhesive Polymers

As used herein, the term “mucoadhesive” refers to natural, synthetic or semi-synthetic materials that adhere to mucosal surfaces, e.g., that adhere to surfaces upon wetting or hydration Mucoadhesive polymers suitable for use as pharmaceutically acceptable carriers for the compositions described herein are known. For example, U.S. Pat. No. 6,562,363 describes mucoadhesive polymers (also referred to as “bioadhesive polymers) that are useful in the compositions described herein. The entirety of U.S. Pat. No. 6,562,363 is incorporated herein by reference.

Examples of suitable mucoadhesive polymers include, but are not limited to, carbomers, carboxyethylene polymers, and/or polyacrylic acids (such as CARBOPOL® 980 or 940 NF, 981 or 941 NF, 1382 or 1342 NF, 5984 or 934 NF, ETD 2020, 2050, 934P NF, 971P NF, 974P NF, Noveon AA-1 USP); poly(2-hydroxyethyl methacrylate) (PHEMA), poly(methacrylic acid), polyethylene oxides, poloxamers, cellulose derivatives such as hydroxypropylmethylcellulose (HPMC), ethylhydroxyethylcellulose (EHEC), carboxymethylcellulose (CMC), salts of carboxymethylcellulose, hydroxypropylcellulose (HPC), hydroxyethylcellulose (HEC), hydroxyethylmethylcellulose, arabic gum, shiraz gum, karaya gum, xanthan gum, guar gum, gellan gum, alginates (e.g., sodium alginate), hyaluronates (e.g., hyaluronic acid, sodium hyaluronate), polyvinylpyrrolidones, chitosans (e.g., chitosan, thiolated chitosan), pectin, gelatin, polyvinyl alcohols, and veegum.

Polyvinylpyrrolidone or “PVP” refers to a polymer, either a homopolymer or copolymer, containing N-vinylpyrrolidone as the monomeric unit. Typical PVP polymers are homopolymeric PVPs and the copolymer vinyl acetate vinylpyrrolidone. The homopolymeric PVPs are known to the pharmaceutical industry under a variety of designations including Povidone, Polyvidone, Polyvidonum, Polyvidonum soluble, and Poly(1-vinyl-2-pyrrolidone). The copolymer vinyl acetate vinylpyrrolidone is known to the pharmaceutical industry as Copolyvidon, Copolyvidone, and Copolyvidonum. The term “soluble” when used with reference to PVP means that the polymer is soluble in water and generally is not substantially cross-linked, and has a molecular weight of less than about 2,000,000. See generally, Buhler, KOLLIDON®: POLYVINYLPRYRROLIDONE FOR THE PHARMACEUTICAL INDUSTRY, BASF Aktiengesellschaft (1992).

The compositions described herein may include one or more types of mucoadhesive polymers, including mixtures or blends of two or more mucoadhesive polymers. In this regard, different mucoadhesive polymers may be associated with different properties. For example, mucoadhesive polymers that dissolve in water (or in the environment of the gastroinstestinal tract) such as PVP and PVA polymers can be used to prepare relatively thicker patches due to their high solids content, and exhibit good compatibility with enhancers (a)-(e) described above, even at relatively high concentrations of enhancer, and exhibit good adhesiveness. Mucoadhesive polymers that dissolve slowly in water (or in the environment of the gastroinstestinal tract) such as CMC, HEC, HPC and poly(acrylic acid) also can be used to prepare relatively thicker patches due to their moderate solids content, and exhibit good compatibility with permeation enhancers (a)-(e) described above, even at relatively high concentrations of enhancer, and exhibit good adhesiveness. Mcoadhesive polymers that swell in water (or in the environment of the gastroinstestinal tract) such as chitosan, alginate, guar gum, and karaya gum can be used to prepare relatively thinner patches due to their low solids content, but exhibit limited compatibility with permeation enhancers (a)-(e) described above, such that it may be advantageous to use a relatively lower amount of such an enhancer with such mucoadhesive polymers, and also tend to form patches that are more brittle and exhibit less adhesiveness. Other mucoadhesive polymers, such as those that do not dissolve or swell in water (or in the environment of the gastroinstestinal tract), such as HPMC and PHEMA, also can be used to prepare relatively thicker patches due to their moderate solids content, and exhibit good compatibility with permeation enhancers (a)-(e) described above, even at relatively high concentrations of enhancer, and exhibit good adhesiveness. Thus, a skilled artisan may select suitable mucoadhesive polymers and combinations thereof to formulate a bremelanotide delivery compositions and/or patch with desired properties.

In some embodiments, the pharmaceutically acceptable carrier of a mucoadhesive layer of a transmucosal drug delivery patch includes one or more mucoadhesive polymers selected from poly(2-hydroxyethyl methacrylate), poly(methacrylic acid), polyacrylic acids, thiolated polyacrylic acids, hydroxypropylmethylcellulose (HPMC), ethylhydroxyethylcellulose (EHEC), carboxymethylcellulose (CMC), sodium carboxymethylcellulose, hydroxypropylcellulose (HPC), hydroxyethylcellulose (HEC), hydroxyethylmethylcellulose, arabic gum, shiraz gum, karaya gum, xanthan gum, guar gum, gellan gum, sodium alginate, sodium hyaluronate, polyvinylpyrrolidone, chitosan, thiolated chitosan, pectin, and polyvinyl alcohol. In some embodiments, the pharmaceutically acceptable carrier includes one or more mucoadhesive polymers selected from poly(2-hydroxyethyl methacrylate), poly(methacrylic acid), polyacrylic acids, thiolated polyacrylic acids, hydroxypropylmethylcellulose (HPMC), ethylhydroxyethylcellulose (EHEC), carboxymethylcellulose (CMC), sodium carboxymethylcellulose, hydroxypropylcellulose (HPC), hydroxyethylcellulose (HEC), hydroxyethylmethylcellulose, arabic gum, shiraz gum, karaya gum, xanthan gum, guar gum, gellan gum, sodium alginate, sodium hyaluronate, polyvinylpyrrolidone, chitosan, thiolated chitosan, pectin, and polyvinyl alcohol.

In specific embodiments, the pharmaceutically acceptable carrier comprises one or more mucoadhesive polymers selected from HPMC, HEC, HPC, PHEMA, PVP, PVA, and karaya gum. In some embodiments, the mucoadhesive polymer comprises HPMC. In some embodiments, the mucoadhesive polymer comprises HEC. In some embodiments, the mucoadhesive polymer comprises HPC. In some embodiments, the mucoadhesive polymer comprises PHEMA. In some embodiments, the mucoadhesive polymer comprises PVP. In some embodiments, the mucoadhesive polymer comprises PVA. In some embodiments, the mucoadhesive polymer comprises a blend of PVP and karaya gum.

### Pressure-Sensitive Adhesive Polymers

As used herein, the term “pressure-sensitive adhesive” refers to a viscoelastic material which adheres instantaneously to most substrates with the application of very slight pressure and remains permanently tacky. A polymer may be a pressure-sensitive adhesive within the meaning of the term as used herein if it has the properties of a pressure-sensitive adhesive per se or may functions as a pressure-sensitive adhesive by admixture with tackifiers, plasticizers or other additives. The term pressure-sensitive adhesive also includes mixtures of different polymers and mixtures of polymers, such as polyisobutylenes (PIB), of different molecular weights, wherein each resultant mixture is a pressure-sensitive adhesive. In the last case, the polymers of lower molecular weight in the mixture are not considered to be “tackifiers,” said term being reserved for additives which differ other than in molecular weight from the polymers to which they are added.

Pressure-sensitive adhesive polymers suitable for use as pharmaceutically acceptable carriers for the compositions described herein are known. For example, U.S. Pat. No. 6,024,976 describes pressure-sensitive adhesive polymers and polymer blends that are useful in the compositions described herein. The entirety of U.S. Pat. No. 6,024,976 is incorporated herein by reference. In general, suitable polymers include solvent-based pressure-sensitive adhesive polymers, hot melt pressure-sensitive adhesive polymers, and grafted adhesives, which may be used alone or in combinations, mixtures or blends. Examples of suitable pressure-sensitive adhesive polymers include acrylic polymers, silicone polymers, rubbers, gums, polyisobutylenes, polyvinylethers, polyurethanes, styrene block copolymers, styrene/butadiene polymers, polyether block amide copolymers, ethylene/vinyl acetate copolymers, and vinyl acetate based adhesives.

### Other Pharmaceutically Acceptable Carriers and Other Components

As noted above, the compositions described herein be in any form suitable for topical, transdermal or transmucosal delivery, including transmucosal delivery through gastrointestinal mucosa after oral administration. In addition to the patch embodiments described above, in some embodiments the composition is formulated in a liquid or semi-solid non-finite form for topical administration (such as a solution, gel, emulsion, cream, plaster, etc.), or is formulated in a solid non-finite form (e.g., powder or granules, optionally provided in capsules) for transmucosal delivery through oral, buccal, sublingual, vaginal, rectal and/or gastrointestinal mucosa.

The pharmaceutically acceptable carrier can be selected based on the specific dosage form being prepared and the intended route or administration and site of application. In some embodiments, a pharmaceutically acceptable carrier may include any one or more components typically used as pharmaceutical carriers, excipients, surfactants, diluents, disintigrants, buffers, stabilizers, and the like, such as any one or more of those described, for example, in Remington's Pharmaceutical Sciences (Remington's Pharmaceutical Sciences, 20th ed., Mack Publishing Company, Easton, Pa., USA, 2000).

The following dosage forms are described by way of example illustrating various dosage forms and pharmaceutically acceptable carriers, and should not be construed as limiting.

Non-finite solid dosage forms suitable for oral, buccal, sublingual, rectal or vaginal administration, may be in the form of powders or granules, optionally further provided in the form of tablets, capsules, gelcaps, caplets, and sachets, for example. These can be prepared, for example, by mixing bremelanotide (or a pharmaceutically acceptable salt thereof) and one or more penetration enhancers with at least one pharmaceutically acceptable carrier suitable for the specific solid dosage form being prepared, such as pharmaceutically acceptable carriers for tablets or capsules, such as one or more of sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Additionally or alternatively, any solid dosage forms can contain other ingredients that confer one or more desired properties, such as an inactive diluent, lubricants such as magnesium stearate, preservatives such as paraben or sorbic acid, anti-oxidants such as ascorbic acid, tocopherol or cysteine, disintegrating agents, binders, thickeners, buffers, sweeteners, flavoring agents, perfuming agents, etc. Any of these dosage forms also may be provided with suitable coating materials known in the art, such as cosmetic coatings, enteric coatings, delayed release coatings, controlled release coatings, etc. In some embodiments, the composition in non-finite solid form (e.g., in the form of a powder or granules) is provided in a unit dose pharmaceutical dosage form, such as a tablet, capsule, gelcaps, caplets, or sachet, including unit dose oral pharmaceutical dosage forms.

Liquid dosage forms suitable for topical topical (including buccal and sublingual), oral, rectal or vaginal administration may be in the form of emulsions, syrups, elixirs, suspensions, gels, and solutions. These can be prepared, for example, by mixing bremelanotide (or a pharmaceutically acceptable salt thereof) and one or more penetration enhancers with at least one pharmaceutically acceptable carrier suitable for the specific dosage form and route of delivery, such as one or more of water, an oil, and an alcohol. Additionally or alternatively, any liquid dosage forms can contain other ingredients that confer one or more desired properties, such as surfactants, suspending agents, emulsifying agents, preservatives, anti-oxidants, thickeners, buffers, sweeteners, flavoring agents, perfuming agents, etc.

Specific exemplary pharmaceutically acceptable liquid carriers and excipients include, but are not limited to, peanut oil, sesame oil, cottonseed oil, corn oil and olive oil; esters of fatty acids such as ethyl oleate, isopropyl myristate, fatty acid glycerides and acetylated fatty acid glycerides; alcohols, such as, but not limited to, ethanol, isopropyl alcohol, hexadecyl alcohol, glycerol and propylene glycol; ethers, such as but not limited to, poly(ethyleneglycol); and petroleum hydrocarbons such as mineral oil and petrolatum.

Semi-solid dosage forms for topical (including buccal and sublingual), oral, transdermal, rectal or vaginal administration include ointments, pastes, creams, lotions, and gels. These can be prepared, for example, by mixing bremelanotide (or a pharmaceutically acceptable salt thereof) and one or more penetration enhancers with at least one pharmaceutically acceptable carrier suitable for the specific dosage form. Pharmaceutically acceptable carriers for ointments, pastes, creams, lotions, and gels include those described above for liquid dosage forms, and also hydrophilic polymers, gelling polymers, swelling polymers, excipients such as animal and vegetable fats, oils, waxes, paraffins, starch, tragacanth, cellulose derivatives, polyethylene glycols, silicones, bentonites, silicic acid, talc and zinc oxide, and mixtures thereof.

### Backing

In some embodiments, a mucoadhesive or transdermal patch as described herein is provided with a backing, such as a backing layer or coating on the drug-containing layer that protects the drug(s) from the external environment or internal environment of use (e.g., the gastrointestinal environment) and/or prevents loss of drug(s) to the environment and/or diffusion of the drug(s) in unintended directions (e.g., into the gastrointestinal environment rather than through gastrointestinal mucosa as intended). In some embodiments, the backing is a drug-impermeable backing. By “drug-impermeable” backing means a backing that is substantially impermeable to the drug formulated in the drug-containing layer such that no substantial amount of drug loss through the backing is observed while the backing is intact, as indicated by less than about 10% diffusing through the backing when the drug-containing layer is adhered to a skin or mucosas surface while the backing is intact. Thus, in some embodiments, a backing protects the drug-containing layer from the environment and prevents loss of the drug and/or release of other components to the environment during use.

Materials suitable for use in a backing as described herein are known in the art and can comprise films (for backing layers) or volatile solutions (for backing coatings) of polymeric materials such as ethylcellulose, polymethacrylate esters, polyester, polyethylene, vinyl acetate resins, ethylene/vinyl acetate copolymers, polymethacrylates, methacrylic acid-methyl methacrylate copolymers, polyvinyl chloride, polyurethane, and the like, and, for backing layers, metal foils, non-woven fabric, cloth, and commercially available laminates. A typical backing has a thickness in the range of 1 to 2000 micrometers. Other suitable backing materials include commercially available backings films, such as polyester-based laminate backings such as 3M Scotchpak® backings (3M. St. Paul, Minn.) as well as polymethacrylate-based backings utilizing EUDRAGIT® methacrylic acid-methyl methacrylate copolymers (Evonik Industries). Suitable backings can be selected based on the intended site of application, i.e., buccal versus gastrointestinal versus skin.

In some embodiments, the backing is provided by a coating, such as a spray coating. In some such embodiments, a liquid backing composition, such as a solution comprising a polymer (such as ethlycellose, polymethacrylate esters, polyester, polyethylene, ethylene/vinyl acetate copolymers, polymethacrylates, methacrylic acid-methyl methacrylate copolymers, polyvinyl chloride, polyurethane, etc.) in a volatile solvent, is applied (e.g., sprayed, cast, or applied) onto a prepared mucoadhesive layer, and then the solvent is evaporated, such as by drying. In some such embodiments, the resulting backing is not a separate layer or pre-formed film, but rather is integrally formed on a surface of the mucoadhesive layer. In some such embodiments, the backing is provided by application of a desired coat weight of the backing composition to the prepared mucoadhesive layer.

In some embodiments particularly suitable for mucoadhesive patches, the backing is a dissolvable backing such as those described in U.S. Pat. No. 7,276,246. Dissolvable backings may comprise one or more hydrophilic polymers that dissolve in physiological fluids, such as tears, saliva or gastrointestinal fluids. Examples of such hydrophilic polymers include, but are not limited to, gelatin, caseine, pectin, agar, chitosan, carrageenan, starch, dextran, methylcellulose, calcium carboxymethylcellulose, sodium carboxymethyl cellulose, cross-linked polymer of sodium carboxymethylcellulose (e.g., croscarmellose sodium), microcrystalline cellulose, ethylcellulose, hydroxyethylcellulose, hydroxypropylcellulose, hydroxypropylmethylcellulose, hydroxypropylmethylcellulose phthalate, cellulose ethers, cellulose acetate, cellulose acetate phthalate, other cellulose derivatives, polyvinyl alcohol, polyvinylpyrrolidone (PVP), cross-linked povidone, other vinyl polymers and copolymers, guar gum, poloxamer, polyethylene glycol, polyethylene oxide, polyacrylic acid, polyethers, alkoxy polymers, sodium alginate, xanthan gum, other natural hydrogels, and hydrogels derived from natural products. In addition to the hydrophilic polymer, a solvent or plasticizer may be included in a dissolvable backing, such as to make the backing more flexible. Examples of suitable plasticizers include, but are not limited to, glycerin, propylene glycol, polyethylene glycol, polypropylene glycol, sugar alcohols, and corn syrup.

In some embodiments, the backing extends to the outer perimeter of the drug-containing layer, i.e., it is coterminous with the active surface area of the drug-containing layer. In some embodiments using a backing layer, the backing layer extends beyond the outer perimeter of the drug-containing layer in one or more or all directions, i.e., it overhangs the active surface area of the drug-containing layer in one or more or all directions. In such embodiments, backing layer may prevent drug from leaking out of the side the drug-containing layer. This helps ensure that the drug passes through the skin or mucosal surface as opposed to diffusing to the surrounding environment.

As noted above, providing a patch with a backing offers several advantages. For example, as noted above, the backing promotes unidirectional delivery of the drug through the skin or mucosal surface. The backing protects the drug-containing layer from the environment of the application site. Without this protection a patch may suffer from drug loss and/or degradation. The backing also contributes to the high concentration gradient of drug at the skin or mucosal site of application provided by a patch as described herein, which in turn promotes effective drug delivery.

### Release Liners

In some embodiments, a mucoadhesive or transdermal patch as described herein is provided with a release liner, typically located adjacent the opposite side of the drug-containing layer as compared to the backing. When present, the release liner is removed from the patch prior to use to expose the drug-containing layer prior to application. Materials suitable for use as release liners are known in the art and commercially available, and include silicone-coated polyethylene, polypropylene, polyester, and polystyrene release liners sold under the PRIMELINER™ brand as supplied by Loparex LLC (Cary, N.C.) and 3M Scotchpak™ fluoropolymer-coated polyester release liners supplied by 3M (St. Paul, Minn.).

### Pharmaceutical Capsules Containing Mucoadhesive Patches

In some embodiments, one or more mucoadhesive patches as described herein is provided in a pharmaceutical capsule. For example, a pharmaceutical capsule may contain one or more, 10 or more, 20 or more, 30 or more, 40 or more, 50 or more, 60 or more, 70 or more, 80 or more, 90 or more, or 100 or more transmucosal drug delivery patches according to any one or more of the embodiment described herein. See FIG. 1C.

An oral pharmaceutical capsule may be designed to deliver the transmucosal mucoadhesive drug delivery patches to the gastrointestinal mucosa, including the stomach or other specific region of the gastrointestinal tract, such as by being made from a material that erodes, dissolves or disintegrates in the gastrointestinal tract, such as the stomach or other specific region of the gastrointestinal tract, such as the duodenum, jejunum, ileum, terminal ileum, etc. A rectal or vaginal pharmaceutical capsule may be designed to deliver the transmucosal mucoadhesive drug delivery patches to the rectal or vaginal mucosa, respectively.

Pharmaceutical capsules are known in the art and commercially available. For example, an oral pharmaceutical capsule may be made from material that erodes, dissolves or disintegrates in the gastrointestinal tract, such as a gelatin, a polysaccharide material (including a naturally-occurring or modified polysaccharide, such as carrageenan, starch, cellulose, hypromellose, including naturally-occurring and modified variants thereof). The pharmaceutical capsule may be of any size capable of containing the intended number and size of patches, such as capsule ranging in size from a size 9 capsule to a size 00 capsule, or larger.

In some embodiments, an oral pharmaceutical capsule is provided with a coating that protects the capsule until it reaches the target region of the gastrointestinal tract, such as an enteric coating, pH-dependent coating, pH-independent (time dependent) coating, controlled release coating, and/or delayed release coating that protects the capsule until it reaches the target region of the gastrointestinal tract.

### Targeted Delivery Oral Pharmaceutical Dosage Forms

In some embodiments, the composition in solid non-finite form (such as powder or granule form) is formulated to deliver the composition to the gastrointestinal mucosa, including the stomach or other specific region of the gastrointestinal tract, such as by being provided with a coating that erodes, dissolves or disintegrates in the gastrointestinal tract, such as the stomach or other specific region of the gastrointestinal tract, such as the duodenum, jejunum, ileum, terminal ileum, etc., or by being provided in a dosage form (such as a tablet, capsule, caplet, gelcap, or the like) made of a material or provided with a coating that erodes, dissolves or disintegrates in the gastrointestinal tract.

In some embodiments, the composition or oral pharmaceutical dosage form is provided with a coating that protects the dosage form (e.g., prevents release of the drug) until it reaches the target region of the gastrointestinal tract, such as an enteric coating, pH-dependent coating, pH-independent (time-dependent) coating, controlled release coating and/or delayed release coating that protects the dosage form until it reaches the target region of the gastrointestinal tract.

### Manufacturing Methods

The compositions described herein may be prepared by methods known in the art. Preparation of the compositions in non-finite form may include blending (mixing) the bremelanotide (or pharmaceutically acceptable salt thereof) and penetration enhancer, any carrier components being used and any other optional components being used, and forming the composition into a suitable dosage form as described above, such as filling into sachets or capsules, forming into tablets, or formulating into solutions, suspensions, emulsions, gels, etc. Preparation of the compositions in flexible, finite (patch) form may include blending (mixing) the adhesive polymer(s) and, as needed, other excipients with an appropriate amount of penetration enhancer and bremelanotide (or pharmaceutically acceptable salt thereof) in the presence of an appropriate solvent, such as water and/or a volatile organic solvent, casting the wet blend onto a solid support (such as a release liner or backing), followed by evaporation of the solvent(s) at appropriate drying conditions. Optionally, a backing layer (if being used, and not already used as a support) can be applied (e.g., laminated) to the dried mucoadhesive layer. Optionally, a backing coating (if being used) can be applied (e.g., sprayed, cast, or applied) to the dried mucoadhesive layer.

In accordance with any embodiments of the mucoadhesive patches described herein, the total thickness of the patch maybe from about 10 m to about 1 mm, or any value or range including and in between any of these values. In accordance with any embodiments of the of the mucoadhesive patches described herein, the patch may have a diameter (or, if not circular, a longest diameter) of from about 100 m to about 20 mm, or any value or range including and in between any of these values. In embodiments where the patches are filled into a capsule, the diameter of the patches can be any size that can fit within the capsule.

In accordance with any embodiments of the transmucosal drug delivery patch described herein, the patch may have a size of, for example from about 0.0003 cm² to about 3 cm². Thus, the size of the transmucosal delivery patch may be about 0.0003 cm², about 0.0005 cm², about 0.001 cm², about 0.003 cm², about 0.005 cm², about 0.01 cm², about 0.03 cm², about 0.05 cm², about 0.1 cm², about 0.15 cm², about 0.2 cm², about 0.25 cm², about 0.3 cm², about 0.35 cm², about 0.4 cm², about 0.45 cm², about 0.5 cm², about 0.55 cm², about 0.60 cm², about 0.65 cm², about 0.70 cm², about 0.75 cm², about 0.80 cm², about 0.85 cm², about 0.9 cm², about 0.95 cm², about 1 cm², about 1.2 cm², about 1.4 cm², about 1.6 cm², about 1.8 cm², about 2.0 cm², about 2.5 cm², about 3 cm², or any value or range including and in between any of these values.

In accordance with any embodiments of the transdermal patches described herein, the total thickness of the patch maybe from about 10 m to about 5 mm, or any value or range including and in between any of these values. In accordance with any embodiments of the of the transdermal patches described herein, the patch may have a diameter (or, if not circular, a longest diameter) of from about 1 cm² to about 140 cm², or any value or range including and in between any of these values.

An exemplary general method for preparing a unit final product of a transmucosal delivery patch as described in any embodiment herein is as follows:
1. Appropriate amounts of one or more mucoadhesive polymers, solvent(s) and/or co-solvent(s), and optional excipient(s) are combined and thoroughly mixed together in a vessel.
2. The drug and permeation enhancer are added to the mixture and agitation is carried out until the drug and permeation enhancer is uniformly mixed therein.
3. The composition is transferred to a coating operation where it is coated onto a support (such as a release liner or backing material) at a controlled specified thickness. The coated composition is then dried at ambient temperature or passed through an oven in order to drive off all processing solvents.
4. Optionally. the mucoadhesive layer is brought into contact with a backing layer or coated with a liquid backing material and dried.
5. Appropriate size and shape transmucosal drug delivery patches are die-cut.
6. Optionally, appropriate numbers of transmucosal drug delivery patches are filled into pharmaceutical capsules.

The order of steps, the amount of the ingredients, and the amount and time of agitation or mixing may be important process variables which will depend on the specific mucoadhesive polymers, drugs, permeation enhancers, solvents and/or co-solvents, and optional excipients used, but these factors can be adjusted by those skilled in the art. The order in which each method step is performed can be changed if needed without detracting from the invention.

An exemplary general method for preparing a unit final product of a transdermal delivery patch as described in any embodiment herein is as follows:
1. Appropriate amounts of one or more pressure-sensitive adhesive polymers, solvent(s) and/or co-solvent(s), and optional excipient(s) are combined and thoroughly mixed together in a vessel.
2. The drug and permeation enhancer are added to the mixture and agitation is carried out until the drug and permeation enhancer is uniformly mixed therein.
3. The composition is transferred to a coating operation where it is coated onto a release liner at a controlled specified thickness. The coated composition is then dried at ambient temperature or passed through an oven in order to drive off all processing solvents.
4. Optionally. the pressure-sensitive adhesive layer is brought into contact with a backing layer or coated with a liquid backing material and dried.
5. Appropriate size and shape transdermal drug delivery patches are die-cut.

The order of steps, the amount of the ingredients, and the amount and time of agitation or mixing may be important process variables which will depend on the specific adhesive polymers, drugs, permeation enhancers, solvents and/or co-solvents, and optional excipients used, but these factors can be adjusted by those skilled in the art. The order in which each method step is performed can be changed if needed without detracting from the invention.

### Therapeutic Methods

The compositions described herein can achieve a transdermal and/or transmucosal flux of bremelanotide that is sufficient to have a therapeutic effect. As used herein, “flux” (also called “permeation rate”) is defined as the absorption of a drug through mucosal tissue, and is described by Fick's first law of diffusion:

J=−D(dCm/dx)

where J is the flux in g/cm²/sec, D is the diffusion coefficient of the drug through the skin or mucosa in cm²/sec and dCm/dx is the concentration gradient of the drug across the skin or mucosa.

The compositions described herein are useful in methods for administering bremelanotide, including topically, transdermally or transmucosally administering bremelanotide for local or systemic effect. Thus, described herein are methods for administering bremelanotide or pharmaceutically acceptable salt thereof to a subject in need thereof, comprising orally administering a composition as described herein, including a patch or capsule containing patches as described herein to a subject in need thereof. Also described herein are methods for administering bremelanotide or pharmaceutically acceptable salt thereof to a subject in need thereof, comprising topically, transdermally or transmucosally administering a composition as described herein or a patch as described herein by applying the composition/patch to skin or mucosa of a subject in need thereof.

In some embodiments, the administration comprises oral, transdermal, transmucosal, vaginal, rectal, or ophthalmic administration. In some embodiments, the administration comprises administration to one or more of the oral, ocular, nasal, buccal, vaginal, rectal, or gastrointestinal mucosa of the subject. As noted above, a composition may be administered or applied orally for delivery to the oral, buccal or gastrointestinal mucosa.

The methods described herein are useful for treating any disease or condition in the subject that is treatable by bremelanotide. In some embodiments, the subject is suffering from or at risk of developing one or more of Hypoactive Sexual Desire Disorder (HSDD), erectile dysfunction, inflammatory disease, hemorrhagic shock, reperfusion injury (ischemia), aphthous stomatitis, arthritis, and acute kidney failure as may occur after surgery. In some embodiments, the methods comprise administering a therapeutically effective amount of bremelanotide for the condition being treated. Such therapeutically effective amounts can be determined by the skilled artisan by approaches known in the art, such as by extrapolation from animal studies and from dose-ranging studies in human clinical trials.

### Examples

The examples below are provided to illustrate aspects the present technology, and should in no way be construed as limiting the scope of the invention described herein.

### Aqueous Formulations

An aqueous composition comprising bremelanotide and an enhancer as described herein was prepared (Example 1) and transdermal delivery was assessed and compared with a control composition that did not include a penetration enhancer (Control 1). The components of each composition are provided in Table 1.

TABLE 1
Aqueous Bremelanotide Compositions

| Formulation | Example 1 | Control 1 |
|---|---|---|
| Bremelanotide Acetate | 20 mg | 20 mg |
| Sodium N-(8-[2- | 125 mg | — |
| hydroxybenzoyl]amino)caprylate | | |
| (SNAC) | | |
| Water | 1 mL | 1 mL |

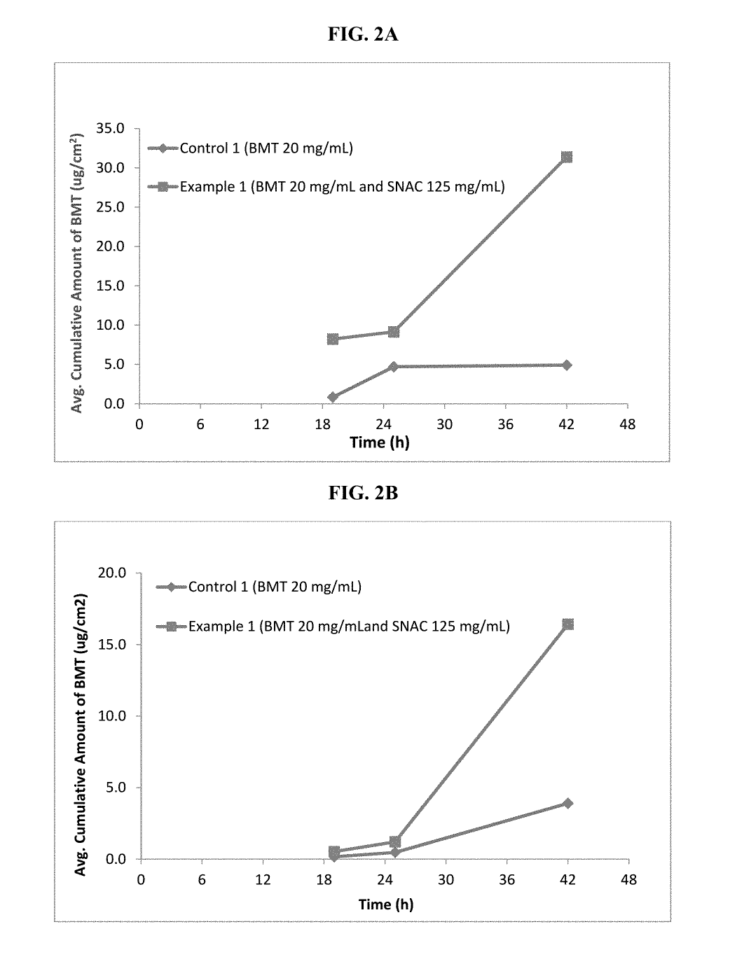

These compositions were tested in vitro in a modified diffusion cell type setup with skin tissue from two different human donors (Donor 1 and Donor 2, respectively). Receptor medium was collected at the times indicated in FIGS. 2A-B, and bremelanotide flux was determined. As illustrated in FIGS. 2A-B, Example 1 exhibited significant transdermal delivery of bremelanotide as compared to Control 1. (Results are reported as the average from n=3 samples.)

### Transbuccal Delivery of Bremelanotide In Vitro

Transbuccal deliver of bremelanotide was assessed using Example 1 and Control 1 above, as well as transmucosal drug delivery patches with the formulations provided in Table 2. To prepare the transmucosal drug delivery patches, mucoadhesive polymer, sodium N-(8-[2-hydroxybenzoyl]amino)caprylate (SNAC), and bremelanotide acetate were mixed well in a vial using water or water/ethanol as a processing solvent, casted and coated onto a release liner, and dried overnight at room temperature (about 21° C.). The dried mucoadhesive layers were coated with an ethylcellulose solution to provide a backing coating. Patches were punched out for testing.

TABLE 2
Transmucosal Drug Delivery Patch Mucoadhesive Layers for In Vitro
Transbuccal Study

| | Example 2 | Example 3 |
|---|---|---|
| Formulation | (% w/w) | (% w/w) |
| Bremelanotide Acetate | 7.70 | 3.39 |
| Hydroxypropylmethyl | 55.38 | — |
| Cellulose (HPMC) | | |
| Polyvinylpyrrolidone | — | 49.80 |
| (PVP) | | |
| Karaya Gum | — | 9.96 |
| SNAC | 36.92 | 36.85 |

Each component in Table 2 is reported as dry weight percent of the total dry weight of the mucoadhesive layer in the particular patch.

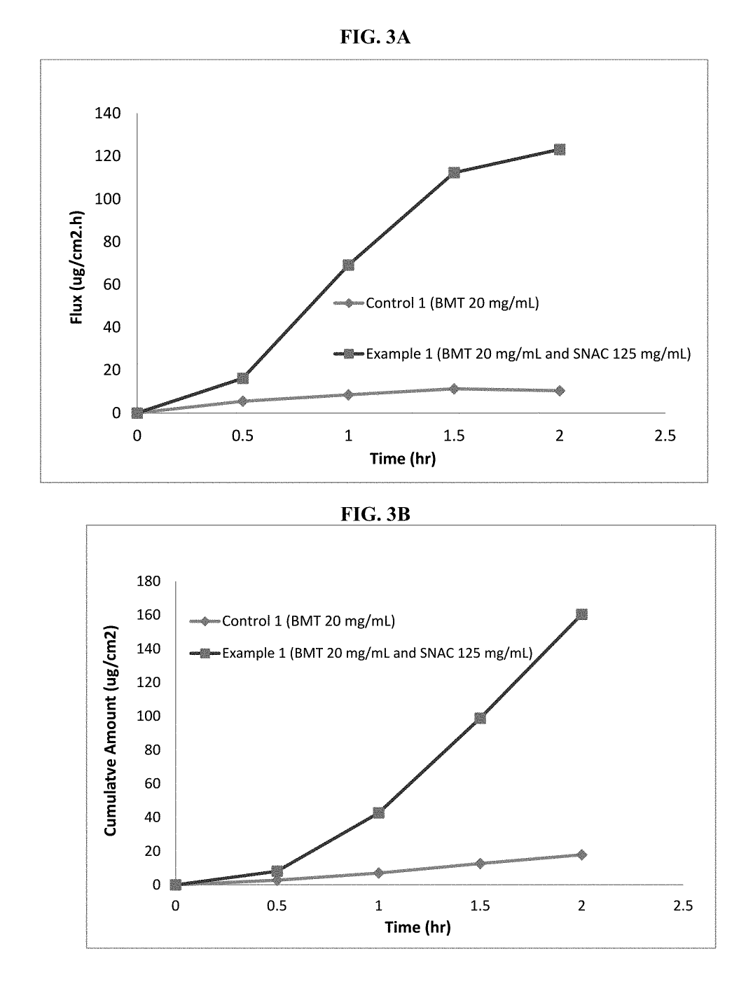

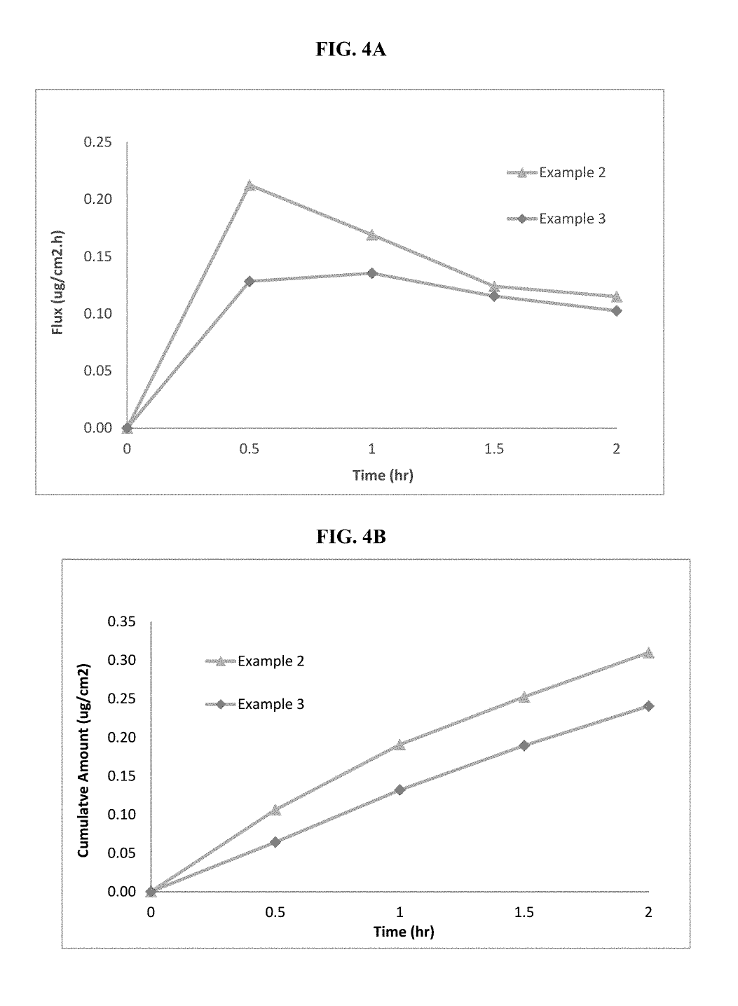

An in vitro buccal tissue model in a modified diffusion cell type setup was utilized to assess the flux of bremelanotide across buccal mucosal tissue. The compositions of Example 1 and Control 1 were applied to the tissue, while the transmucosal drug delivery patches (Examples 2 and 3) were applied to the tissue with the mucoadhesive layer in contact with the tissue. Receptor medium was was collected at the times indicated in FIGS. 3A-B and FIGS. 4A-B and and bremelanotide flux was determined. As illustrated by FIGS. 3A-B (where “BMT” stands for bremelanotide), Example 1 again exhibited significant transdermal delivery of bremelanotide as compared to Control 1. Patch Examples 2 and 3 achieved delivery of drug, although at lower levels than the aqueous composition of Example 1. Patch Examples 2 and 3 exhibited good adhesion to the buccal tissue. (Results are reported as the average from n=3 samples.) While the particular formulation in the patches (Examples 2 and 3) exhibited less drug delivery than the aqueous composition of Example 1, it should be noted that the in vitro model does not simulate the dynamic fluid environment surrounding mucosal tissue in vivo. In such an environment, the aqueous composition of Example 1 may not be in contact with mucosal surfaces for a sufficient time period to deliver the target amount of drug through the mucosa. On the other hand, mucoadhesive patches would maintain constant with mucosal surfaces in vivo. Thus, aqueous compositions may exhibit reduced drug delivery in vivo as compared to this in vitro study. Moreover, mucoadhesive patches could provide targeted delivery to specific sites of action (e.g., gastrointestinal mucosa) that may be poorly available to aqueous compositions.

### Oral Capsules Containing Bremelanotide Compositions

Tables 3 and 4 set forth the formulations used to prepare oral capsules containing bremelanotide compositions. For Example 4, a powder blend of bremelanotide acetate and SNAC was prepared and filled into capsules. For Examples 5-15, transmucosal drug delivery patches were prepared as described above, provided with an ethylcellulose backing coating, punched out, and filled into capsules. For Control 2, bremelanotide acetate in powder form was filled into capsules.

TABLE 3
Summary of Capsules Containing Bremelanotide

| | | Formulation | | | | | | |
|---|---|---|---|---|---|---|---|---|
| | | Control 2 | Ex. 4 | Ex. 5 | Ex. 6 | Ex. 7 | Ex. 8 | Ex. 9 |
| Component | BA | 100 | 22.41 | 7.97 | 7.80 | 3.90 | 7.80 | 7.80 |
| (Weight %) | PVP | — | — | 53.69 | 55.92 | 38.25 | 35.00 | 14.08 |
| | Karaya Gum | — | — | 10.74 | 11.18 | 7.65 | 7.00 | 2.82 |
| | PHEMA | — | — | — | — | — | — | — |
| | SNAC | — | 77.59 | 27.60 | 25.10 | 50.20 | 50.20 | 75.30 |

| | | | | | | | |
|---|---|---|---|---|---|---|---|
| Patch Size (cm²) | N/A | N/A | 0.0314 | 0.0314 | 0.0314 | 0.0314 | 0.0314 |
| Number of Patches | N/A | N/A | 20 | 22 | 22 | 22 | 22 |
| Capsule Size | 9 | 9 | 9 | 9 | 9 | 9 | 9 |
| Total SNAC | 0 | 4.5 | 4.5 | 4.5 | 9.0 | 9.0 | 13.5 |
| (mg/capsule) | | | | | | | |
| Total BA (mg/capsule) | 1.3 | 1.3 | 1.3 | 1.4 | 0.7 | 1.4 | 1.4 |

BA = Bremelanotide Acetate
PHEMA = Poly(2-hydroxyethylmethacrylate)

TABLE 4
Summary of Capsules Containing Bremelanotide (cont.)

| | | Formulation | | | | | |
|---|---|---|---|---|---|---|---|
| | | Ex. 10 | Ex. 11 | Ex. 12 | Ex. 13 | Ex. 14 | Ex. 15 |
| Component | BA | 7.80 | 3.90 | 7.80 | 7.80 | 3.90 | 2.54 |
| (Weight %) | PVP | — | — | — | — | — | — |
| | Karaya Gum | — | — | — | — | — | — |
| | PHEMA | 67.10 | 45.90 | 42.00 | 16.90 | 68.20 | 64.56 |
| | SNAC | 25.10 | 50.20 | 50.20 | 75.30 | — | 27.90 |
| | n-DMS | — | — | — | — | 27.90 | 5.00 |

| | | | | | | |
|---|---|---|---|---|---|---|
| Patch Size (cm²) | 0.0314 | 0.0314 | 0.314 | 0.314 | 0.0314 | 0.0314 |
| Number of Patches | 22 | 22 | 22 | 22 | 22 | 18 |
| Capsule Size | 9 | 9 | 9 | 9 | 9 | 9 |
| Total Enhancer | 4.5 | 9.0 | 9.0 | 13.5 | 5.0 | 5.2 |
| (mg/capsule) | | | | | | |
| Total BA (mg/capsule) | 1.4 | 0.7 | 1.4 | 1.4 | 0.7 | 0.4 |

n-DMS = n-dodecyl-β-D-maltoside

### In Vivo Delivery of Bremelanotide by Oral Capsules (Rats)

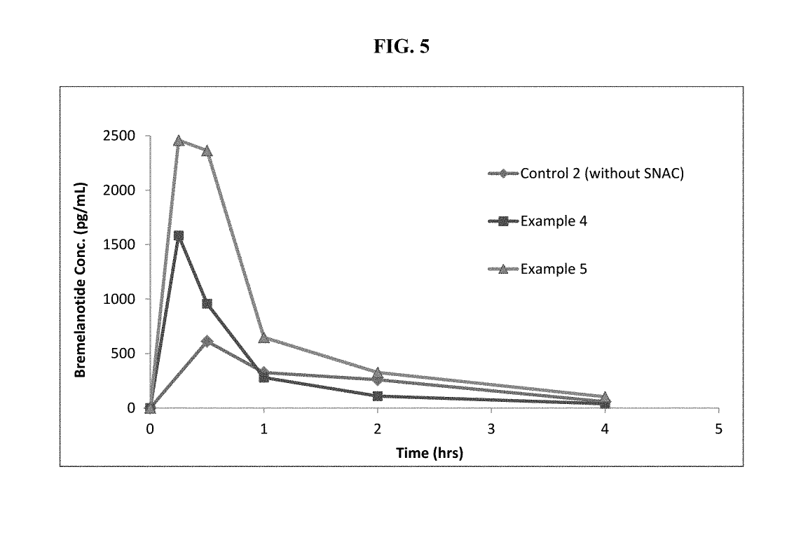

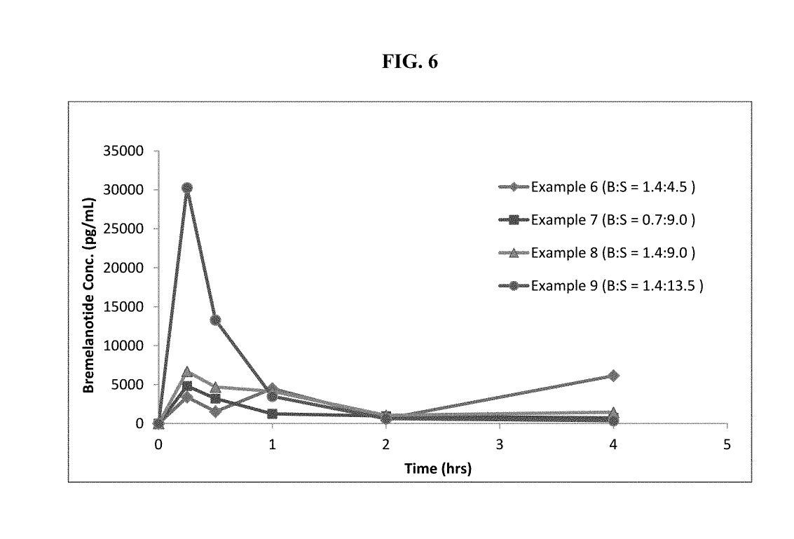

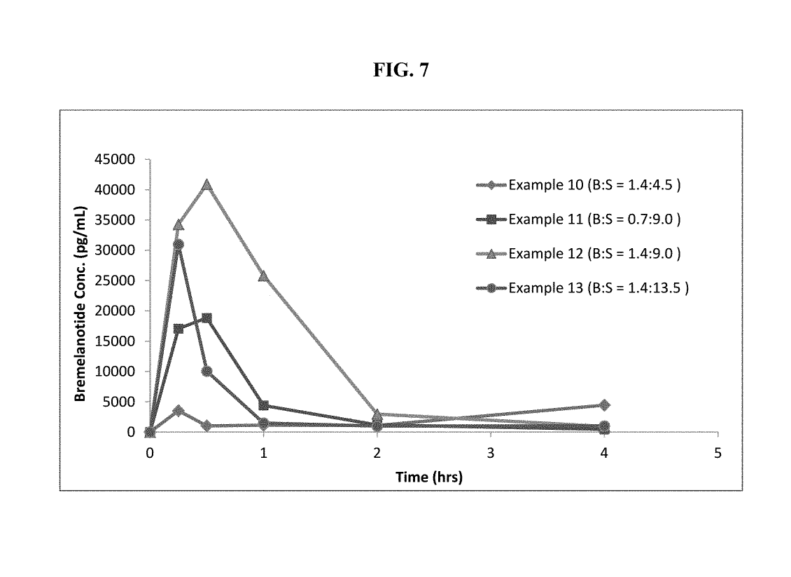

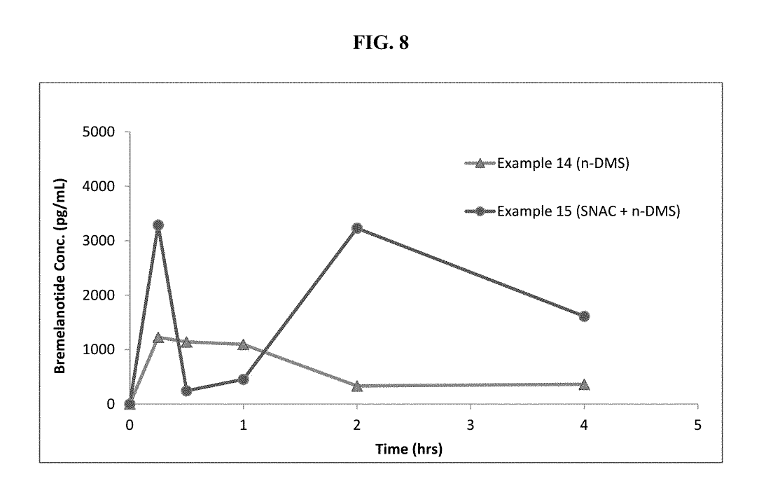

The capsules of Examples 4-15 and Control 2, described above, were tested in vivo in a rat model. Capsules were administered by oral gavage administration using a dosing tool, followed by administration of 0.3 mL of water. Blood samples were drawn until 5 hours after administration and assayed for bremelanotide concentration as a function of time. FIGS. 5-8 illustrate the blood levels (pg/ml) of bremelanotide achieved (reported as the average from n=2 or 3 rats per formulation tested). Of note, all compositions formulated with an enhancer as described herein achieved significant blood levels and significantly improved bremelanotide delivery as compared to Control 2, including greater area under the curve (“AUC”). As shown in FIG. 5, the patch of Example 5 achieved greater drug delivery than the powder blend compositions of Example 4. The results illustrated in FIGS. 6 and 7 indicate that increasing the relative amount of penetration enhancer results in increased bremelanotide delivery. The results illustrated in FIG. 8 indicate that n-DMS as well as mixtures of SNAC and n-DMS are effective enhancers for bremelanotide through the GI tract.

Overall, these data show successful delivery of drugs by the bremelanotide compositions and patches described herein.

### In Vivo Delivery of Bremelanotide by Oral Capsules (Dogs)

Table 5 sets forth the formulations used to prepare oral capsules containing bremelanotide compositions. For Example 16, a powder blend of bremelanotide acetate and SNAC was prepared and filled into capsules. For Examples 17-18, transmucosal drug delivery patches were prepared as described above, provided with an ethylcellulose backing coating, punched out and filled into capsules. For Example 17, the capsules were provided with an enteric coating.

TABLE 5
Summary of Capsules Containing Bremelanotide

| Formulation | | Ex. 16 | Ex. 17 | Ex. 18 |
|---|---|---|---|---|
| Component | BA | 6.25 | 3.21 | 3.21 |
| (Weight %) | PVP | — | — | — |
| | Karaya | — | — | — |
| | Gum | | | |
| | PHEMA | — | 48.69 | 48.69 |
| | SNAC | 93.75 | 48.10 | 48.10 |

| | | | |
|---|---|---|---|
| Patch Size (cm²) | N/A | 0.5 | 0.5 |
| Number of Patches | N/A | 24 | 24 |
| Capsule Size | 000 | 000-EC | 000 |
| Total SNAC | 150 | 150 | 150 |
| (mg/capsule) | | | |
| Total BA (mg/capsule) | 10 | 10 | 10 |

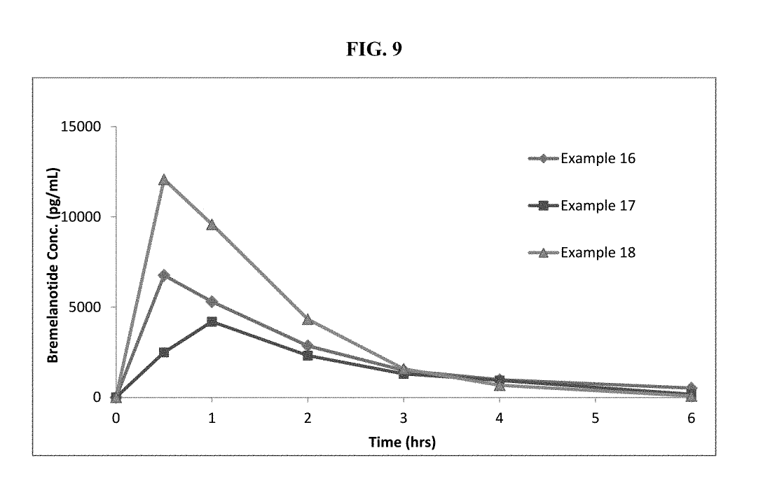

The capsules of Examples 16-18 were tested in vivo in dogs (n=5 dogs per formulation tested). Capsules were administered by oral gavage administration, followed by administration of water. Blood samples were drawn until 6 hours after administration and assayed to determine bremelanotide concentration as a function of time. FIG. 9 illustrates the blood levels (pg/ml) of bremelanotide achieved. Example 18 (containing patches) exhibited significantly better Cₘₐₓ and AUC than the capsule containing a physical blend of the same drug and enhancer components (Example 16). Example 17 with enteric-coated capsules containing patches achieved less drug delivery than the non-enteric-coated capsules containing patches, and also less drug delivery than the capsules containing the powder blend. However, Example 17 did provide significant blood levels of bremelanotide. The results show successful delivery of drugs by the bremelanotide compositions described herein, even in larger animals such as dogs.

What is claimed is:

1. A pharmaceutical composition for transdermal or transmucosal delivery of bremelanotide, comprising bremelanotide or a pharmaceutically acceptable salt thereof and a penetration enhancer, wherein the penetration enhancer comprises one or more selected from:
(a) a compound of Formula I 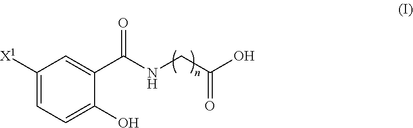
wherein X¹ is H or Cl and n is an integer from 6 to 21, or a pharmaceutically acceptable salt thereof;
(b) an N-substituted bis-3,6-[4-aminobutyl]-2,5-diketopiperazine or a pharmaceutically acceptable salt thereof;
(c) an alkyl glucoside or a pharmaceutically acceptable salt thereof;
(d) lauroyl-L-carnitine; and
(e) dimethyl palmitoyl ammonio propanesulfonate or a pharmaceutically acceptable salt thereof.

2. The composition of claim 1, wherein the penetration enhancer comprises one or more selected from N-(8-[2-hydroxybenzoyl]amino)caprylic acid (SNAC), N-(5-chlorosalicyloyl)-8-aminocprylic acid (5-CNAC), N-(10[2-hydroxybenzoyl]amino)decanoic acid (SNAD), bis-3,6(4-fumarylaminobutyl)-2,5-diketopiperazine, n-dodecyl-β-D-maltoside, lauroyl-L-carnitine, dimethyl palmitoyl ammonio propanesulfonate, and pharmaceutically acceptable salts of any one or more thereof.

3. The composition of claim 1, wherein the penetration enhancer comprises N-(8-[2-hydroxybenzoyl]amino)caprylic acid (SNAC).

4. The composition of claim 1, wherein the penetration enhancer comprises n-dodecyl-β-D-maltoside.

5. The composition of claim 1, wherein the penetration enhancer constitutes from about 0.1% to about 99.9% by weight of the composition.

6. The composition of claim 1, wherein the weight ratio of penetration enhancer to bremelanotide or pharmaceutically acceptable salt thereof is from about 999:1 to about 1:999.

7. The composition of claim 1, further comprising a pharmaceutically acceptable carrier for topical, transdermal or transmucosal administration comprising one or more selected from a mucoadhesive polymer, a pressure-sensitive adhesive polymer, and water.

8. The composition of claim 7, wherein the pharmaceutically acceptable carrier comprises a mucoadhesive polymer comprising one or more selected from hydroxypropylmethylcellulose (HPMC), polyvinylpyrrolidone, poly(2-hydroxyethyl methacrylate) (PHEMA), carbomers, carboxyvinyl polymers, poly(methacrylic acid), polyacrylic acid, thiolated poly(acrylic acid), ethylhydroxyethylcellulose (EHEC), carboxymethylcellulose (CMC), a salt of carboxymethylcellulose, hydroxypropylcellulose (HPC), hydroxyethylcellulose (HEC), hydroxyethylmethylcellulose, polyvinyl alcohol, karaya gum, arabic gum, shiraz gum, xanthan gum, guar gum, gellan gum, alginate, sodium alginate, chitosan, thiolated chitosan, hyaluronate, sodium hyaluronate, pectin, gelatin, and veegum.

9. The composition of claim 7, wherein the pharmaceutically acceptable carrier comprises a mucoadhesive polymer comprising one or more selected from HPMC, HEC, HPC, PHEMA, PVP, PVA and karaya gum.

10. A transdermal or transmucosal patch comprising the composition of claim 7, in a flexible, finite form wherein the pharmaceutically acceptable carrier comprises a mucoadhesive polymer or a pressure-sensitive adhesive polymer.

11. The patch of claim 10, wherein the composition is provided as a drug-containing layer and the patch further comprises a backing.

12. The patch of claim 11, wherein the backing comprises one or more selected from ethylcellulose, polyester, polyethylene, vinyl acetate resins, ethylene/vinyl acetate copolymers, polymethacrylates, methacrylic acid-methyl methacrylate copolymers, polyvinyl chloride, polyurethane, foil, non-woven fabric, and cloth.

13. The patch of claim 11, wherein the backing is coterminous with the perimeter of the drug-containing layer.

14. The patch of claim 11, wherein the backing comprises a backing layer that extends beyond the outer perimeter of the drug-containing layer in one or more or all directions.

15. The patch of claim 11, wherein the backing is a dissolvable backing that dissolves in the environment of the mouth or gastrointestinal tract.

16. The patch of claim 10, wherein the composition is substantially free of plasticizers other than enhancers (a) through (e).

17. The patch of claim 10, wherein the patch is a transmucosal patch, wherein the pharmaceutically acceptable carrier comprises a mucoadhesive polymer.

18. The patch of claim 10, wherein the patch is a transdermal patch, wherein the pharmaceutically acceptable carrier comprises a pressure-sensitive adhesive polymer.

19. The patch of claim 10, comprising an amount of bremelanotide or pharmaceutically acceptable salt thereof effective to deliver a therapeutically effective amount thereof.

20. An oral pharmaceutical capsule containing a composition according to claim 1

21. The capsule of claim 20, wherein the capsule contains a plurality of patches, each comprising the composition in a flexible, finite form comprising a pharmaceutically acceptable carrier comprising a mucoadhesive polymer or a pressure-sensitive adhesive polymer.

22. The capsule of claim 20, wherein the capsule releases the bremelanotide or pharmaceutically acceptable salt thereof in the gastrointestinal tract.

23. The capsule of claim 20, comprising an amount of bremelanotide or pharmaceutically acceptable salt thereof effective to deliver a therapeutically effective amount thereof.

24. A method for administering bremelanotide or pharmaceutically acceptable salt thereof to a subject in need thereof, comprising orally administering a composition according to claim 1 to a subject in need thereof.

25. A method for administering bremelanotide or pharmaceutically acceptable salt thereof to a subject in need thereof, comprising topically, transdermally or transmucosally administering a composition according to claim 1 to a subject in need thereof.

26. The method of claim 25, wherein the administration comprises administration to one or more of the oral, ocular, nasal, buccal, vaginal, rectal, or gastrointestinal mucosa of the subject.

27. The method of claim 24, wherein the subject is suffering from or at risk of developing Hypoactive Sexual Desire Disorder (HSDD), erectile dysfunction, inflammatory disease, hemorrhagic shock, reperfusion injury (ischemia), aphthous stomatitis, arthritis, and/or acute kidney failure.
